(19) United States
(12) Patent Application Publication
(10) Pub. No.: US 2025/0177122 A1
(43) Pub. Date: Jun. 5, 2025

(54) OPHTHALMIC LENS WITH REDUCED CHROMATIC ABERRATION

(71) Applicant: Alcon Inc., Fribourg (CH)

(72) Inventors: Zhiguang Xu, Colleyville, TX (US); Shinwook Lee, Arlington, TX (US); Wei Lu, Colleyville, TX (US); Sangyeol Lee, North Richland Hills, TX (US); Xin Hong, Fort Worth, TX (US)

(21) Appl. No.: 18/953,526

(22) Filed: Nov. 20, 2024

Related U.S. Application Data
(60) Provisional application No. 63/660,648, filed on Jun. 17, 2024.
(60) Provisional application No. 63/606,350, filed on Dec. 5, 2023.

(51) Int. Cl. A61F 2/16

(52) U.S. Cl. CPC A61F 2/1618; A61F 2240/001

(57) ABSTRACT

An ophthalmic lens defining an optical axis can include: a plurality of annular regions extending in a radial direction away from the optical axis, including a corresponding plurality of echelettes, wherein the plurality of echelettes forms an echelette profile, wherein each the echelette profile includes a staircase profile, wherein each echelette includes a staircase step, wherein, for each staircase step, there are M phase-shift units arranged vertically, wherein M is an integer, wherein each phase-shift unit has a phase height configured to shift the phase of a reference frequency of light by approximately N*2*π, where N is an integer, and wherein the staircase profile forms an irregular staircase.

### CROSS REFERENCE TO RELATED APPLICATIONS

The present application claims priority to and the benefit of U.S. Prov. Nos. 63/606,350 and 63/660,648, filed on Dec. 5, 2023 and Jun. 17, 2024 respectively, the entireties of which are incorporated by reference herein.

### BACKGROUND

Generally, this application relates to ophthalmic lenses, such as multifocal intraocular lenses (IOLs). A multifocal IOL focuses light into multiple focal regions. One type of multifocal IOL is a diffractive IOL. A multifocal IOL can be used for refractive lens exchange or cataract surgery to replace the natural lens in the eye and correct a patient's refractive errors (e.g., nearsightedness). Other types of ophthalmic lenses include contact lenses and spectacle lenses. While the benefits of ophthalmic lenses, such as multifocal IOLs, are known, improvements to optical designs continue to address challenges with present designs, such as chromatic aberrations, in order to improve outcomes and benefit patients.

### SUMMARY

According to embodiments, an ophthalmic lens defining an optical axis includes: a plurality of annular regions extending in a radial direction away from the optical axis, including a corresponding plurality of echelettes, wherein the plurality of echelettes forms an echelette profile, wherein the echelette profile includes a staircase profile, wherein each echelette includes a staircase step, wherein, for each staircase step, there are M phase-shift units arranged vertically, wherein M is an integer, wherein each phase-shift unit has a phase height configured to shift the phase of a reference frequency of light by approximately N*2*π, where N is an integer, and wherein the staircase profile forms an irregular staircase. A differential in a number of phase-shift units between adjacent staircase steps in the staircase profile may be less than or equal to −2. A differential in a number of phase-shift units between adjacent staircase steps in the staircase profile may be zero. A differential in a number of phase-shift units between adjacent staircase steps in the staircase profile may be more than or equal to +2. The ophthalmic lens may be an intraocular lens (IOL). The IOL may be configured to be implanted into a patient's eye through a hole in the patient's cornea having a diameter of no larger than 4 mm. The IOL may include a material having a Young's Modulus of no greater than 300 MPa. The IOL may be a quadrifocal IOL.

According to embodiments, a method is disclosed for constructing a multifocal intraocular lens (IOL) defining a plurality of focal regions corresponding to focal points for a plurality of diffractive orders, wherein the multifocal IOL includes a plurality of annular regions formed in a medium and extending in a radial direction away from an optical axis, wherein the plurality of annular regions include a plurality of echelettes, wherein each echelette includes a staircase step including M phase-shift units, wherein M is an integer, wherein each phase-shift unit has a thickness approximately equal to N wavelengths for a reference frequency of light, wherein N is an integer, wherein each echelette includes a diffractive unit, wherein the arrangement of the plurality of echelettes defines an echelette profile, wherein the arrangement of the plurality of staircase steps defines a staircase profile, the method includes: determining, for each of a plurality of candidate echelette profiles, a characteristic for each of the plurality of focal regions; determining, for each characteristic in a given candidate echelette profile, a visual acuity value; combining each visual acuity value in each of the given ones of the candidate echelette profiles to form a combined score for each of the candidate profiles; selecting, from the plurality of candidate echelette profiles, a selected echelette profile according to the combined score for the selected echelette profile; and fabricating the multifocal IOL using the selected echelette profile. At least two of the plurality of candidate echelette profiles may have different staircase profiles. At least one of the plurality of candidate echelette profiles may include an irregular staircase. At least two of the candidate echelette profiles may have different diffractive profiles. At least two of the candidate echelette profiles may have different staircase profiles. Each combined score may be a sum of the visual acuity values for given ones of the candidate echelette profiles. The plurality of diffractive orders do not include the first diffractive order. The plurality of diffractive orders may include only the zeroth, second, and third diffractive orders. The step of determining, for each characteristic in a given candidate echelette profile, a visual acuity value further may include assessing a modulation transfer function for each of the plurality of focal regions.

According to embodiments, an ophthalmic lens defining an optical axis and a plurality of focal points includes: a plurality of annular regions extending in a radial direction away from the optical axis, including a corresponding plurality of echelettes formed in a medium, wherein the plurality of echelettes form an echelette profile, wherein each echelette includes a staircase step, wherein the plurality of staircase steps define a staircase profile, wherein each staircase step includes M phase-shift units, wherein M is an integer, wherein each phase-shift unit has a thickness approximately equal N wavelengths for a reference frequency of light through the medium, where N is an integer, wherein the staircase profile includes an irregular staircase, and wherein the irregular staircase is selected from a plurality of candidate staircase profiles according to a combined visual acuity score for a plurality of focal regions corresponding to the plurality of focal points. A differential in a number of phase-shift units between adjacent steps in the irregular staircase may be less than or equal to −2. A differential in a number of phase-shift units between adjacent steps in the irregular staircase may be zero. A differential in a number of phase-shift units between adjacent steps in the irregular staircase may be more than or equal to +2. The ophthalmic lens may be an intraocular lens (IOL). The IOL may be configured to be implanted into a patient's eye through a hole in the patient's cornea having a diameter of no larger than 4 mm. The IOL may include a material having a Young's Modulus of no greater than 300 MPa. The IOL may be a quadrifocal IOL. The combined visual acuity score may be a sum of visual acuity values for the plurality of focal regions. The plurality of focal points may correspond to a plurality of diffractive orders, wherein the plurality of diffractive orders may not include the first diffractive order. The plurality of diffractive orders may include only the zeroth, second, and third diffractive orders. Each visual acuity value may be determined according to a modulation transfer function for each corresponding focal region.

### BRIEF DESCRIPTION OF SEVERAL VIEWS OF THE DRAWINGS

FIG. 1 illustrates an IOL focusing light.

FIG. 2 illustrates an IOL with an echelette profile diffracting and focusing light.

FIG. 3 illustrates an IOL, according to embodiments.

FIG. 4 illustrates an echelette profile with an irregular staircase, according to embodiments.

FIG. 5 illustrates an echelette profile with an irregular staircase, according to embodiments.

FIG. 6 illustrates an IOL with an echelette profile having an irregular staircase and diffracting and focusing light, according to embodiments.

FIG. 7 illustrates simulated visual acuity scores for an IOL with an echelette profile including an irregular staircase, where the simulated visual acuity scores are at different simulated distances, according to embodiments.

FIG. 8 illustrates simulated visual acuity scores for different IOLs having different echelette profiles at different simulated distances, according to embodiments.

FIG. 9 illustrates a combination of simulated visual acuity scores shown in FIG. 8, according to embodiments.

FIG. 10 is a flowchart for a method of constructing a multifocal IOL having an irregular staircase, according to embodiments.

FIG. 11 illustrates an echelette profile having a uniform staircase profile.

The foregoing summary, as well as the following detailed description of certain techniques of the present application, will be better understood when read in conjunction with the appended drawings. For the purposes of illustration, certain techniques are shown in the drawings. It should be understood, however, that the claims are not limited to the arrangements and instrumentality shown in the attached drawings. Furthermore, the appearance shown in the drawings is one of many ornamental appearances that can be employed to achieve the stated functions of the system.

### DETAILED DESCRIPTION

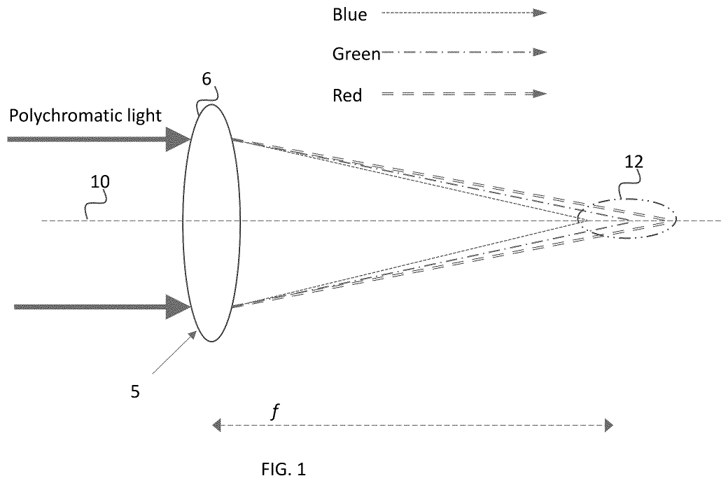

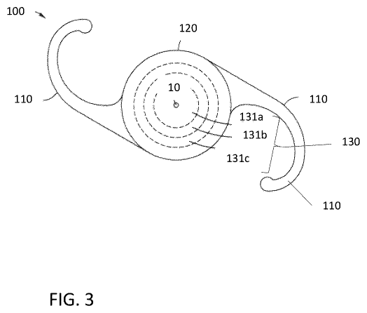

FIG. 1 illustrates an IOL 5 focusing light and the principle of chromatic aberration. While reference is made to IOLs herein, certain embodiments are similarly applicable to other types of ophthalmic lenses, such as contact lenses or spectacle lenses, as will be understood. Generally, the eye receives polychromatic light comprised of photons having different wavelengths (e.g., red, green, and blue). One wavelength for green light has a wavelength in a vacuum of 550 nm. This wavelength is referred to herein as the design wavelength, although other wavelengths may be used for the design wavelength. When polychromatic light is refracted by a lens (e.g., the lens body 6 shown in FIG. 1), different wavelengths travel in different directions. This causes light having different wavelengths to focus at different points. As used herein, the collection of these different points may correspond to or be a focal region, such as focal region 12. A focal region corresponds to the area or volume in which the polychromatic light is focused according to a given diffractive order. A focal region may correspond to focal lengths for wavelengths across the visible light spectrum or a larger spectrum including ultraviolet and/or infrared wavelengths, or only a portion thereof. A focal region may be elliptical, circular, or other shape. A focal region may be two dimensional or three dimensional. According to techniques described herein (see, e.g., FIG. 3), embodiments of IOL 100 and echelette profile 130 reduce the extent of chromatic aberration by reducing the size of focal region(s). While FIG. 1 illustrates a cross-sectional view of an IOL 5, it will be understood that principles described herein have effect in three dimensions as well.

Chromatic aberration, and particularly larger degrees of chromatic aberration, may correspond to lower visual acuity in a patient. Generally, as the effect of chromatic aberration increases, polychromatic light becomes less focused, and a patient perceives a blurrier image. This results in reduced visual acuity.

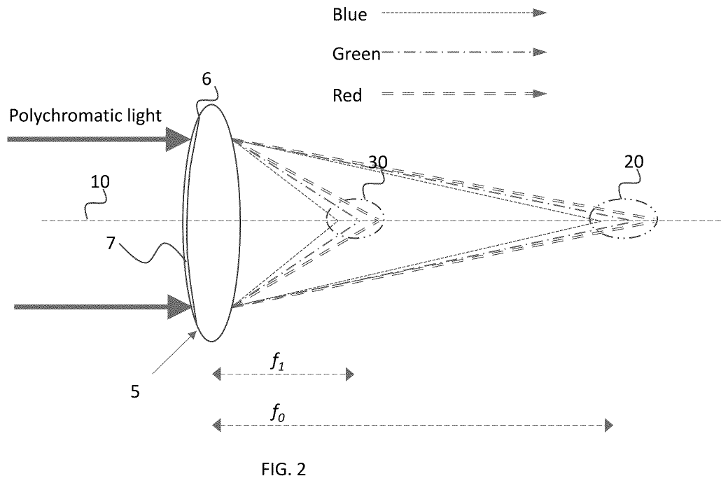

FIG. 2 is similar to FIG. 1, except that an echelette profile 7 (further described below) is included in the IOL 5. Generally, light is diffracted by the IOL 5 having an echelette profile 7 to different locations corresponding to different diffractive orders. FIG. 2 illustrates a focal length f₀ corresponding to the zeroth diffractive order for the design wavelength, and a focal length f₁ corresponding to the first diffractive order for the design wavelength (e.g., 550 nm). The focal lengths depicted are for illustrative purposes only, and may not represent actual focal lengths. Diffracted light for the zeroth diffractive order is focused at a focal region 20. Diffracted light for the first diffractive order is focused at a focal region 30.

According to embodiments, an IOL (such as IOL 100 shown in FIG. 3) may include (be determined at least partially by) three profiles: a base curvature profile; a diffractive profile; and a staircase profile. These profiles may be combined to create the exterior contour of the IOL. Exemplary base curvature profiles and diffractive profiles are disclosed in U.S. Ser. No. 15/498,836, filed on Apr. 27, 2017, the entirety of which is herein incorporated by reference. The combination of the diffractive profile and staircase profile may be arranged in different echelettes arranged over the base curvature profile. The combination of the diffractive profile and staircase profile is referred to herein as an echelette profile. Each echelette may be arranged in an annular region on the exterior contour of the lens (e.g., IOL). The echelettes may be arranged concentrically in annular regions (extend in a radial direction) around the optical axis 10 of the IOL. Each annular region may have substantially the same surface area as other annular regions. The annular regions may be completely or partially annular (e.g., a given echelette does not span completely around the annular region).

Echelettes may be arranged on the anterior of the IOL, the posterior of the IOL, or may be embedded within the IOL. In the case that the echelettes are embedded within the IOL, the echelettes do not extend from the base curvature of the IOL.

FIG. 3 illustrates an elevation view of an IOL 100, according to embodiments. The IOL 100 is configured to be implanted into a patient's eye during a lens implant procedure. During such a procedure, the IOL 100 is in a compressed state (e.g., rolled up) prior to implantation. In order for the IOL 100 to compress in such a manner, it may include a material with a Young's Modulus of no greater than 300 MPa. An opening or incision is formed in the patent's cornea and an opening is formed in the patient's lens capsule. The incision at the cornea may typically have a diameter of no larger than 4 mm. For example, the incision at the cornea may have a diameter between approximately 2 mm and approximately 3.5 mm. The IOL 100 passes through both incisions or openings before entering into the lens capsule, where it decompresses or unfolds. The IOL 100 may readily decompress or unfold once suitably located in situ.

The IOL 100 includes a lens body 120 and two haptic portions 110 coupled to a peripheral, non-optic portion of the lens body 120. The lens body 120 may be fabricated of biocompatible material, such as modified poly (methyl methacrylate) (PMMA), modified PMMA hydrogels, hydroxy-ethyl methacrylate (HEMA), PVA hydrogels, other silicone polymeric materials, and hydrophobic acrylic polymeric materials, for example, AcrySof® and Clareon®, available from Alcon, Inc., Fort Worth, Texas. Such material(s) may suitably compress and decompress as described above. The lens body 120 may have a diameter of between about 4.5 mm and about 7.5 mm, for example, about 6.0 mm. It is noted that the shape and curvatures of the lens body 120 are shown for illustrative purposes only and that other shapes and curvatures are also within the scope of this disclosure. For example, the lens body 120 may have a bi-convex shape. In other examples, the lens body 120 may have a plano-convex shape, a convexo-concave shape, or a plano-concave shape.

A haptic 110 may include hollow radially-extending struts coupled (e.g., glued or welded) to the peripheral portion of the lens body 120 or molded along with a portion of the lens body 120, and thus extend outwardly from the lens body 120 to engage the perimeter wall of the capsular sac of the eye to maintain the IOL 100 in a desired position in the eye. The haptics 110 may be fabricated of biocompatible material, such as modified poly (methyl methacrylate) (PMMA), modified PMMA hydrogels, hydroxy-ethyl methacrylate (HEMA), PVA hydrogels, other silicone polymeric materials, and hydrophobic acrylic polymeric materials, for example, AcrySof® and Clareon®, available from Alcon, Inc., Fort Worth, Texas. The haptics 110 may have radial-outward ends that define arcuate terminal portions. The terminal portions of the haptics 110 may be separated by a length of between about 6 mm and about 22 mm, for example, about 13 mm. The haptics 110 may have a particular length so that the terminal portions create a slight engagement pressure when in contact with the equatorial region of the capsular sac after being implanted. While FIG. 1 depicts one example configuration of the haptics 110, other configurations are contemplated, such as plate haptics.

The depicted IOL 100 in FIG. 3 is a multifocal IOL 100 (having multiple focal points, e.g., bifocal, trifocal, quadrifocal, or pentafocal) that includes a base curvature and echelettes 131a, 131b, 131c formed on the base curvature (e.g., integral with the base curvature). While the echelettes 131a-c are depicted on a surface of the lens body 120 (e.g., anterior or posterior surface), it is contemplated within this disclosure that the echelettes 131a-c may also be positioned within or embedded in an interior of the IOL 100. In the embodiments disclosed herein, the echelettes 131a-c are depicted at the anterior surface of the lens body 120, although the posterior surface of the lens body 120 is contemplated as well. The IOL 100 defines an optical axis 10, around which the echelettes 131a-c may be arranged concentrically. Each echelette 131 may have substantially the same surface area as the other echelettes 131.

As further shown in FIG. 3, successive echelettes 131a, 131b, 131c extend outwardly and radially from the optical axis 10 (radius=0). There may be additional echelettes as will be further described. With this perspective, the first echelette 131a will be closest to the optical axis 10, the second echelette 131b will extend around the first echelette 131a, the third echelette 131c will extend around the second echelette 131b, etc. Further, the echelettes 131 (and components thereof) may be described herein with respect to their “height,” although it is understood that when the ophthalmic lens is in situ or being used by a patient (e.g., a contact lens), the echelettes 131 may generally extend in a horizontal dimension generally along the dimension of the optical axis 10.

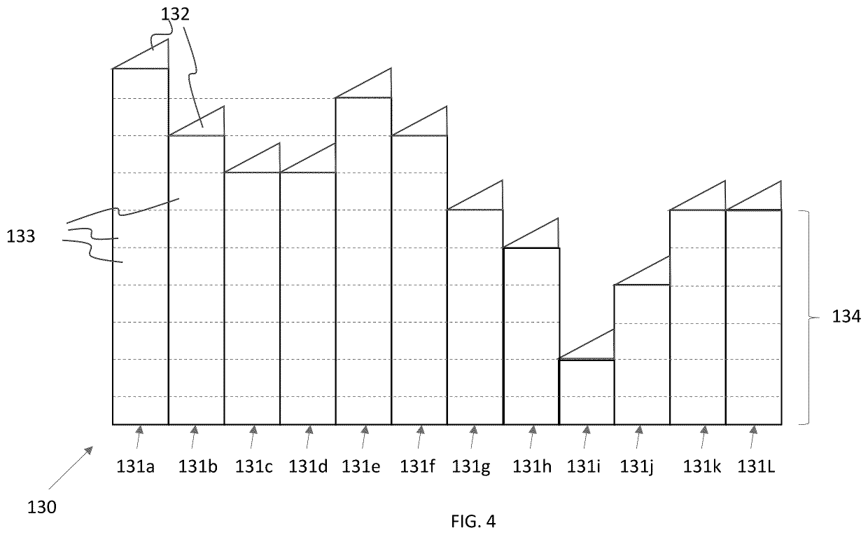

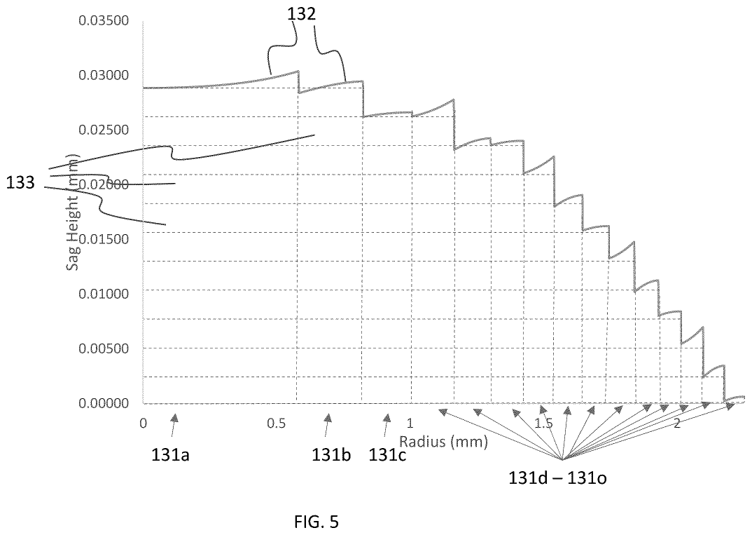

As illustrated in FIGS. 4 and 5, each echelette 131 can include one or more phase-shift units 133 included in a staircase step 134, as well as a diffractive unit 132. The collection of staircase steps 134 in multiple echelettes 131 can be referred to as a “staircase” or having a “staircase profile.”

As illustrated in FIG. 4, the staircase profile is that of an “irregular” staircase (further explained below) and has multiple staircase steps 134 (which are collections of phase-shift units 133), each staircase step 134 corresponding to a different echelette 131. The annular location of each staircase step 134 may correspond to the annular location of each diffractive unit 132. The height of a given staircase step 134 in an echelette 131 is determined in part by the number of phase-shift units 133 in the staircase step 134. A staircase step 134 includes an integer multiple M of phase-shift units 133, where M=0, 1, 2, etc. A phase-shift unit 133 is a section of material (either the same material or a different material that the base lens comprises) that has a height that causes a phase shift in light of a reference wavelength by some integer multiple N of 2π (e.g., 2π, 4π, 6π, etc.). This phase shift is measured between the incoming ray and the outgoing ray of the reference-wavelength light through a phase-shift unit 133. FIG. 5 illustrates another example of an irregular staircase profile.

A phase shift 2π is substantially equal to a single wavelength of light λ (e.g., 550 nm in a vacuum) as it travels through a medium. As used herein, a medium may comprise multiple mediums. While 550 nm is an exemplary wavelength λ, other wavelengths may be used to design embodiments of the ophthalmic lenses described herein.

Using the example of 550 nm light, an index of refraction of the lens material n_{_lens}=1.55, the index of refraction of the surrounding medium n_{_media}=1.336, the equivalent height of one wavelength H is calculated as:

$$H = λ / ( {n}_{\_ lens} - {n}_{\_ media} ) = \sim 2.6 µm$$

Therefore, the height for this type of phase-shift unit 133 is approximately 2.6 μm when N=1. For this example, if N=7, the height of the phase-shift unit 133 is approximately 7*2.6 μm, or 18.2 μm. The height of the phase-shift unit 133 is also referred to herein as phase-shift height, and may be specified in terms of the amount of phase shift (e.g., 2π).

By setting the phase-shift height to be approximately N*H, when the reference light passes across the height of the phase-shift unit 133, the phase of the outgoing reference light will show approximately an integer multiple of a 2π phase shift from the incoming reference light. In a real ophthalmic lens, there may be a tolerance for the phase-shift height, such as +/−30%, +/−10%, or +/−3%, or even less. Because there are M phase-shift units 133 across the height of a given staircase step 134, where M is an integer, the phase-shift height of the staircase step 134 will be M*N*H, approximately a 2π integer multiple.

According to embodiments described herein, and as shown in FIGS. 4 and 5, the staircase may be “irregular.” As used herein, an irregular staircase is one in which there is at least one differential in a given phase-shift height (phase shift by a given one of N*2π) in adjacent staircase steps 134, where the staircase step height differential is at least one of zero, +2 (or greater)*H, or −2 (or less)*H, and wherein this differential is not equal to at least one other phase-shift height differential between other steps in the staircase in adjacent echelettes 131.

One irregular aspect of an irregular staircase can be seen in FIG. 4 by comparing echelette 131c with 131d, in which there is no change in the number of phase-shift units 133. Another irregular aspect of the irregular staircase can be seen by comparing echelette 131d with 131e, where there is an increase in the number of phase-shift units 133 by +2*H. Another irregular aspect of the irregular staircase can be seen by comparing echelette 131h with 131i, where there is a decrease in the number of phase-shift units 133 by −3*H. Irregular aspects can also be seen in the irregular staircase illustrated by FIG. 5, in which the number of phase-shift units 133 in echelettes 131a and 131b is identical, the number of phase-shift units 133 in echelettes 131c and 131d is identical, and the number of phase-shift units 133 in echelettes 131e and 131f is identical.

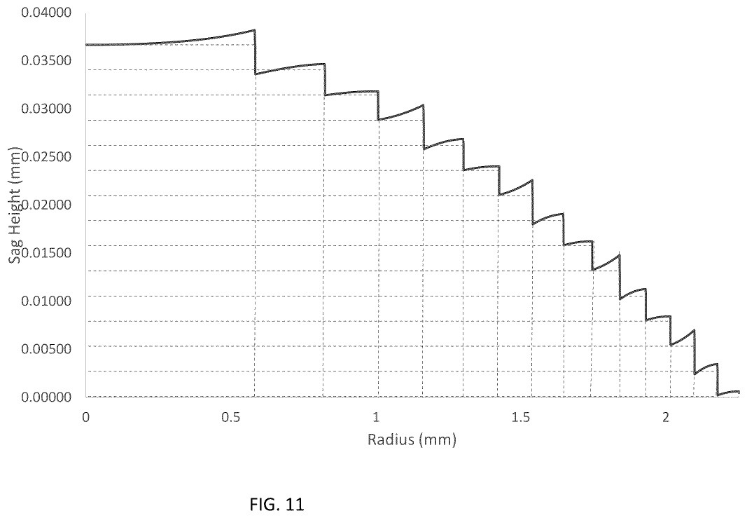

FIG. 11 illustrates an echelette profile having a uniform staircase profile. Using a uniform staircase profile (i.e., not an irregular staircase profile) may be one way to improve achromatization in IOLs. In a uniform staircase profile, the phase shift differential of each staircase step compared to its neighbor is approximately N*2π for a reference wavelength, where N is an integer. This may preserve the monochromatic performance of the IOL (i.e., the monochromatic performance of the lens without the uniform staircase profile) and may improve the IOL's polychromatic performance owing to the uniform staircase profile. However, the level of achromatization owed to the uniform staircase profile may still be insufficient. For example, when applied in multifocal IOLs, the uniform staircase profile may cause over-achromatization for some distances but insufficient achromatization for other distances.

In order to improve achromatization for more than one distance (e.g., every desired distance), an irregular staircase profile can be implemented. As an illustrative example of an irregular staircase profile, if a height of a phase-shift unit 133 is 2.6 μm, then the height of a step in an irregular staircase profile could be 2.6 μm, −2.6 μm, 2*2.6 μm, −2*2.6 μm, 3*2.6 μm, −3*2.6 μm, 0*2.6 μm (zero), etc. Some step heights could be excluded due to factors like manufacturing constraints, for instance, very large step heights or excessive elevations and depressions in the stairway may cause challenge in precise fabrication for some manufacturing techniques.

According to some embodiments, an irregular staircase profile can be non-repeating. In such a case, a given pattern of irregularity within the staircase profile is not repeated in other portions of the staircase profile. For example, if a sequence of the number of staircase steps in the staircase profile for successive echelettes is 1, 2, 3, 0, 0, 1, 2, 3 then the staircase profile is repeating. As another example, if a sequence of staircase steps in the staircase profile for successive echelettes is 1, 2, 3, 1, 2, 3, then the staircase is adjacently repeating. An irregular staircase can be non-repeating for adjacent sequences.

As will be further described, it may be possible to simulate the performance using some or all the possibilities/combinations for each possible staircase profile to determine a preferred irregular staircase profile. In such a way, achromatization at multiple distances or focal points may be improved—e.g., achromatization at each of 0D (diopter), −1.5D, and −2.5D may be simulated.

In certain embodiments, and as shown in FIGS. 4 and 5, one or more or all of the echelettes 131 include a diffractive unit 132. Diffractive units 132 may have a characteristic radial separation to produce constructive interference at characteristic foci. In principle, any diffractive profile that produces constructive interference through phase shifting in interfering zones can be adapted to produce a multifocal diffractive IOL 100. Diffractive units 132 may have different shapes, such as different slope or different shifts between neighboring diffractive units 132 according to the desired performance of the IOL 100. According to embodiments (and as will be further described), different collections of candidate diffractive units 132 can be used to assess performance of an IOL 100 at multiple distances.

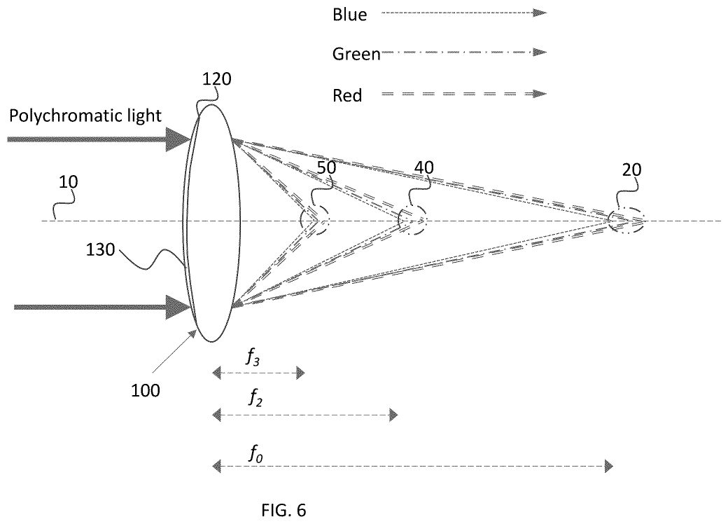

The echelette profile 130 may be used to provide an IOL 100 having two focal lengths (e.g., for near and distance visions), also referred to as a bifocal IOL 100. A bifocal IOL 100 may utilize the zeroth diffractive order for distance vision and the first diffractive order for near vision. In other embodiments, the echelette profile 130 may be used to provide an IOL 100 having three focal lengths for near, intermediate, and distance visions, also referred to as a trifocal IOL 100. A trifocal lens may utilize the zeroth diffractive order for distance vision, the first diffractive order for intermediate vision, and the second diffractive order for near vision. In other embodiments, the echelette profile 130 may be used to provide an IOL 100 having four focal lengths, or a quadrifocal IOL 100. A quadrifocal IOL 100 (an example of which is shown in FIG. 6) may utilize the zeroth diffractive order for distance vision, the first diffractive order for a first intermediate vision (e.g., far-intermediate vision), the second diffractive order for a second intermediate vision (e.g., near-intermediate vision), and the third diffractive order for near vision. Similar principles also apply for pentafocal lenses and beyond.

FIG. 6 illustrates an IOL 100, which is quadrifocal, with an echelette profile 130 focusing light, according to embodiments. FIG. 6 is similar to FIG. 2 in some regards. Notably, however, the size of focal region 20 has been reduced in FIG. 6 in accordance with techniques described herein. The reduction in size of focal region 20 in FIG. 6 as compared to FIG. 2 indicates that the effect of chromatic aberration has been reduced due to the irregular staircase in the IOL 100.

In the example of the quadrifocal IOL 100 of FIG. 6, the first diffractive order is not shown. This illustrates that it may not be preferable to prioritize or afford substantial energy distribution to a given focal region (in this case, energy distributed according to the first diffractive order). Instead, energy distribution is prioritized or allocated to the other diffraction orders (in this case, the zeroth, second, and third diffractive orders). Such distribution may be achieved by designing the diffractive sag profile to achieve the desired energy distribution among diffraction orders such that the given, non-preferred diffractive order (e.g., the first diffractive order) is afforded a lesser energy distribution.

As depicted in FIG. 6, focal regions 20, 40, and 50 are inclusive of or centered at the focal points for diffractive orders. However a focal region 20, 40, or 50 need not be so precisely located. Instead, a given focal region 20, 40, or 50 may correspond to a focal point for a particular diffractive order. The following illustrative example describes how focal regions 20, 40, or 50 may correspond to focal points. Assuming an IOL 100, focal points for the zeroth, second, and third orders correspond to focal lengths of approximately f₀=0D, f₂=−1.65D, and f₃=−2.35D. Focal regions 20, 40, and 50 may be centered at these focal lengths (e.g., on the optical axis 10), respectively. In addition, focal regions 20, 40, or 50 may be located in regions that correspond to the exact focal lengths. For example, a focal region 40 may be centered at −1.5D, instead of at −1.65D for the second diffractive order. As another example, a focal region 50 may be centered at −2.5D, instead of at −2.35D for the third diffractive order.

One reason to assess or define focal regions that are not precisely centered at a diffractive order focal length is because of a mismatch between what types of visual experiences are most common for a patient and how certain clinical vision tests (e.g., legacy clinical vision tests) are performed. Continuing with the example above, the second and third diffractive order focal lengths (f₂, f₃) for the IOL 100 are set to correspond to more frequently encountered distances for a patient in their daily life, where −1.65D is a preferred focal length for an object ˜0.6 m from the IOL 100, and where −2.35D is a preferred focal length for an object ˜0.4 m from the IOL 100. However, certain legacy clinical vision tests are not performed at such focal lengths, and instead are performed at other intervals (e.g., at 0.5D intervals), such as 0D, −0.5D, −1D, −1.5D, −2D, −2.5D, etc. For the second diffractive order, it may be possible to center a focal region 40 at either −1.65D (based on empirical behavior of a typical patient) or −1.5D (a commonly used distance in legacy clinical vision tests), or to center two different focal regions at these different distances. Similarly, for the third diffractive order, it may be possible to center a focal region 50 at either −2.35D (based on empirical behavior of a typical patient) or −2.5D (a commonly used distance in legacy clinical vision tests), or to center two different focal regions at these different distances.

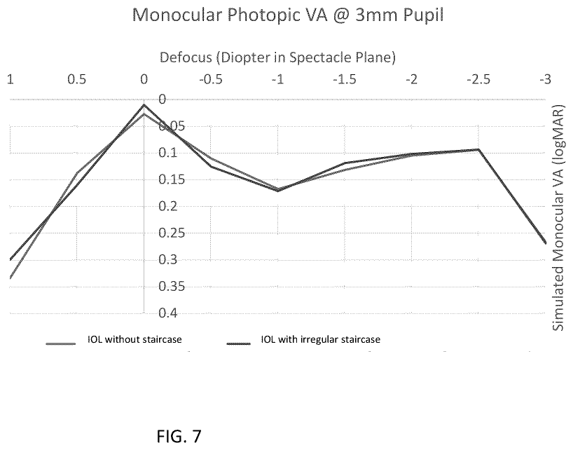

FIG. 7 illustrates simulated the visual acuity via defocus for a multifocal IOL 100 with an irregular staircase simulated at different distances, according to embodiments. The x-axis is the defocus distance from the patient, represented in diopter in spectacle plane. The y-axis represents the visual acuity in unit of logMAR. Lower visual acuity value indicates better vision performance. Visual acuity for both (1) the IOL 100 without the irregular staircase and (2) the IOL 100 with the irregular staircase were simulated at different distances between 1D and −3D, at 0.5D intervals. These simulated visual acuities are examples of performance characteristics. Visual acuity varies between these two curves, at least in part due to altered chromatic aberration. At 0D, visual acuity is substantially improved with the addition of the irregular staircase due to improved chromatic aberration. At −2.5D, the performance of the IOL 100 with the irregular staircase has substantially identical simulated visual acuity than the IOL 100 without the irregular staircase.

Simulation of visual acuity may be performed using a modulation transfer function (MTF). The method of simulation may follow several steps: (1) generate the through-frequency MTF data/curve at each defocus with a valid eye model; (2) calculate the area under the curve (AUC) of the through-frequency MTF curve at each defocus; and (3) use valid coefficients between AUC and visual acuity to achieve the visual acuity at each defocus.

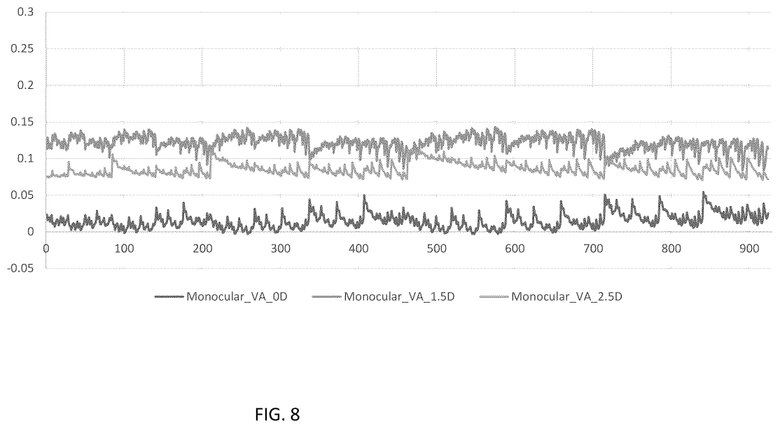

According to embodiments, a selected echelette profile 130 is selected from a group of candidate echelette profiles, where each candidate echelette profile has a unique arrangement of echelettes 131 (either unique staircase profiles and/or unique profiles). FIG. 8 illustrates simulated visual acuity values for different IOLs having different candidate echelette profiles at different focal lengths, according to embodiments. In the particular example of FIG. 8, each location on the x-axis corresponds to a different candidate echelette profile having a unique irregular staircase, with unique arrangements of phase-shift units as compared with the other candidate echelette profiles. The particular arrangement of phase-shift units of a given candidate echelette profile (e.g., the exact number of phase-shift units in particular echelettes and/or the diffractive profile thereof) is not shown.

FIG. 8 shows three curves for the different candidate echelette profiles—one at 0D (corresponding to or equal to the zeroth diffractive order), one at −1.5D (corresponding to the second diffractive order in the example above), and one at −2.5D (corresponding to the third diffractive order in the example above). The y-axis of FIG. 8 has the same scale as the y-axis of FIG. 7. As can be seen, certain candidate echelette profiles perform better or worse at these three different distances. Additional or different curves with other defocus distances are possible, as are different candidate echelette profiles.

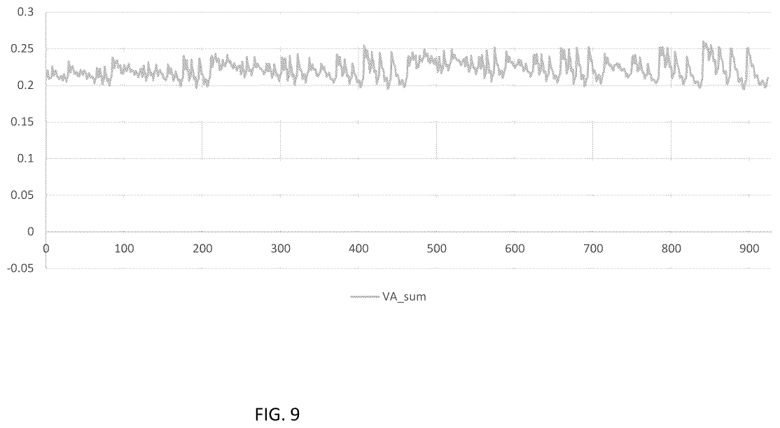

FIG. 9 illustrates a combination of simulated visual acuity scores shown in FIG. 8, according to embodiments. FIG. 9 has similar axes as FIG. 8, but there is only one curve, which is a combination of the three curves in FIG. 8. As shown, candidate echelette profile #900 (on the x-axis) has a relatively high visual acuity score, which corresponds to a poorer overall visual performance. By comparison, candidate echelette profile #400 has a relatively low visual acuity score, which corresponds to an improved overall visual performance. Consequently, candidate echelette profile #400 may be a better candidate for a multifocal IOL than candidate echelette profile #900.

To obtain the curve of FIG. 9, the curves in FIG. 8 were added. However, other ways of combination are possible, such as weighting distances differently. For example, if visual acuity at −2.5D is deemed to be of more importance, that curve in FIG. 8 may be weighted more heavily than the other curves. The combination of curves may be performed in other ways, such as non-linear algorithms.

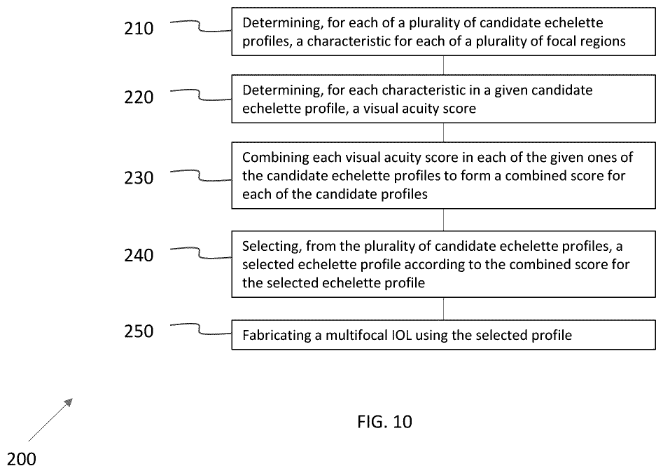

FIG. 10 is a flowchart 200 for a method of fabricating a multifocal IOL 100, according to embodiments. While the flowchart 200 is described with respect to the multifocal IOL 100, the process is not so limited. The flowchart 200 is described with respect to the structural embodiments herein, but is not so limited. Certain steps may be performed with a processor, at least partially, such as steps 210, 220, 230, 240, or 250. These operation(s) may be performed using a computer or other processor having hardware, software, and/or firmware. The various method steps may be performed by modules, and the modules may comprise any of a wide variety of digital and/or analog data processing hardware and/or software arranged to perform the method steps described herein. The modules optionally comprising data processing hardware adapted to perform one or more of these steps by having appropriate machine programming code associated therewith, the modules for two or more steps (or portions of two or more steps) being integrated into a single processor board or separated into different processor boards in any of a wide variety of integrated and/or distributed processing architectures. These methods and systems may employ a tangible media embodying machine-readable code with instructions for performing the method steps described above. Suitable tangible media may comprise a memory (including a volatile memory and/or a non-volatile memory), a storage media (such as a magnetic recording on a floppy disk, a hard disk, a tape, or the like; on an optical memory such as a CD, a CD-R/W, a CD-ROM, a DVD, or the like; or any other digital or analog storage media), or the like.

At step 210, for each of a plurality of candidate echelette profiles, a performance characteristic for each of a plurality of focal regions is determined. Each candidate echelette profile may have a unique staircase profile and/or unique diffractive profile. The focal regions may be selected according to clinical or design preferences. The focal regions may correspond to or be centered around diffractive orders of the multifocal IOL 100. For example, a given focal region may be centered at the second diffractive order (using the example above, −1.65D) or may correspond to the second diffractive order (using the example above, −1.5D). As another example, a given focal region may be centered at the third diffractive order (using the example above, −2.35D) or may correspond to the third diffractive order (using the example above, −2.5D). It may be possible to assess two, three, four, five, six, or more focal regions. As an illustration, if three distances are being assessed (0D, −1.5D, −2.5D) for three different candidate echelette profiles, then a total of nine performance characteristics will be determined.

Some possible candidate echelette profiles may be excluded from the plurality of candidate echelette profiles due to manufacturing limitations or due to quality considerations or other factors.

The performance characteristic of a given candidate echelette profile for a given focal region may represent an assessment of the degree or nature of chromatic aberration in the focal region. Such a performance characteristic may correspond to a function, such as a full-spectrum modulation transfer function (MTF). In human vision, MTF is a measure of the eye's ability to reproduce (or transfer) detail from the object to the image, characterized at different spatial frequencies (i.e., different sizes of details). Full-spectrum MTF combines the MTF information with all the visible wavelengths.

At step 220, for each candidate echelette profile, a visual acuity score is determined. The visual acuity scores may be based on simulations using virtual candidate echelette profiles, and not based on empirical measurements of the performance of given physical candidate echelette profiles. The method of simulation may follow several steps: (1) generate the through-frequency MTF data/curve at each defocus with a valid eye model; (2) calculate the area under the curve (AUC) of the through-frequency MTF curve at each defocus; and (3) use valid coefficients between AUC and visual acuity to achieve the visual acuity at each defocus.

At step 230, each visual acuity score in each of the given ones of the candidate echelette profiles is combined to form a combined score for each of the candidate echelette profiles. For each given candidate echelette profile, the different visual acuity scores can be combined by different methods. For example, the visual acuity scores could be added together with or without weighting. When weighted, more heavily weighted visual acuity scores may correspond to focal regions that are of more importance from a clinical perspective. The visual acuity scores for given candidate echelette profiles may be combined by other methods, such as non-linear algorithms.

At step 240, an echelette profile 130 is selected from the plurality of candidate echelette profiles, according to the combined score for the selected echelette profile 130. For example, a threshold may be predetermined to promote acceptable visual acuity values at multiple defocus distances, and if a combined score is less than the threshold, then the given candidate echelette profile may be suitable for selection.

In an embodiment, the echelette profile 130 is determined by initially starting with a diffractive profile that is constant and combining or adding the diffractive profile to different irregular candidate staircase profiles. According to this embodiment, the aforementioned simulations are performed on different echelette profiles each having the same diffractive profile, but different irregular candidate staircase profiles. In this fashion, each candidate echelette profile has the same diffractive profile. In another embodiment, a plurality of the candidate echelette profiles have the same diffractive profile, and not necessarily every candidate echelette profile has the same diffractive profile.

At step 250, a multifocal IOL 100 is fabricated or constructed using the selected echelette profile 130. The IOL 100 could be fabricated with injection molding technology or some direct cutting technologies.

This disclosure also covers the following clauses which may fully or partly be incorporated into the embodiments.
Clause 1. An ophthalmic lens defining an optical axis, the ophthalmic lens comprising: a plurality of annular regions extending in a radial direction away from the optical axis, including a corresponding plurality of echelettes, wherein the plurality of echelettes forms an echelette profile, wherein the echelette profile includes a staircase profile, wherein each echelette includes a staircase step, wherein, for each staircase step, there are M phase-shift units arranged vertically, wherein M is an integer, wherein each phase-shift unit has a phase height configured to shift the phase of a reference frequency of light by approximately N*2*π, where N is an integer, and wherein the staircase profile forms an irregular staircase.
Clause 2. The ophthalmic lens of claim 1, wherein a differential in a number of phase-shift units between two adjacent staircase steps in the staircase profile is less than or equal to −2.
Clause 3. The ophthalmic lens of claim 1, wherein a differential in a number of phase-shift units between two adjacent staircase steps in the staircase profile is zero.
Clause 4. The ophthalmic lens of claim 1, wherein a differential in a number of phase-shift units between two adjacent staircase steps in the staircase profile is more than or equal to +2.
Clause 5. The ophthalmic lens of claim 1, wherein the ophthalmic lens comprises an intraocular lens (IOL).
Clause 6. The ophthalmic lens of claim 5, wherein the IOL comprises a quadrifocal IOL.
Clause 7. The ophthalmic lens of claim 1, wherein the irregular staircase is a non-repeating staircase.
Clause 8. The ophthalmic lens of claim 7, wherein the irregular staircase is an adjacently non-repeating staircase.
Clause 9. A method for constructing a multifocal intraocular lens (IOL) defining a plurality of focal regions corresponding to focal points for a plurality of diffractive orders, wherein the multifocal IOL includes a plurality of annular regions formed in a medium and extending in a radial direction away from an optical axis, wherein the plurality of annular regions include a plurality of echelettes, wherein each echelette includes a staircase step including M phase-shift units, wherein M is an integer, wherein each phase-shift unit has a thickness approximately equal to N wavelengths for a reference frequency of light, wherein N is an integer, wherein each echelette includes a diffractive unit, wherein the arrangement of the plurality of echelettes defines an echelette profile, wherein the arrangement of the plurality of staircase steps defines a staircase profile, the method comprising: determining, for each of a plurality of candidate echelette profiles, a performance characteristic for each of the plurality of focal regions, wherein each performance characteristic comprises a visual acuity value; combining each visual acuity value in each of the given ones of the candidate echelette profiles to form a combined score for each of the candidate profiles; selecting, from the plurality of candidate echelette profiles, a selected echelette profile according to the combined score for the selected echelette profile; and fabricating the multifocal IOL using the selected echelette profile.
Clause 10. The method of claim 9, wherein at least two of the plurality of candidate echelette profiles have different staircase profiles.
Clause 11. The method of claim 10, wherein at least one of the plurality of candidate echelette profiles includes an irregular staircase.
Clause 12. The method of claim 11, wherein the selected echelette profile comprises an irregular staircase.
Clause 13. The method of claim 12, wherein the irregular staircase is a non-repeating staircase.
Clause 14. The ophthalmic lens of claim 13, wherein the irregular staircase is an adjacently non-repeating staircase.
Clause 15. The method of claim 9, wherein each combined score is a sum of the visual acuity values for given ones of the candidate echelette profiles.
Clause 16. An ophthalmic lens defining an optical axis and a plurality of focal points, the ophthalmic lens comprising: a plurality of annular regions extending in a radial direction away from the optical axis, including a corresponding plurality of echelettes formed in a medium, wherein the plurality of echelettes form an echelette profile, wherein each echelette includes a staircase step, wherein the plurality of staircase steps define a staircase profile, wherein each staircase step includes M phase-shift units, wherein M is an integer, wherein each phase-shift unit has a thickness approximately equal N wavelengths for a reference frequency of light through the medium, where N is an integer, wherein the staircase profile includes an irregular staircase, and wherein the irregular staircase is selected from a plurality of candidate staircase profiles according to a combined visual acuity score for a plurality of focal regions corresponding to the plurality of focal points.
Clause 17. The ophthalmic lens of claim 16, wherein a differential in a number of phase-shift units between adjacent steps in the irregular staircase is less than or equal to −2.
Clause 18. The ophthalmic lens of claim 16, wherein a differential in a number of phase-shift units between adjacent steps in the irregular staircase is zero.
Clause 19. The ophthalmic lens of claim 16, wherein a differential in a number of phase-shift units between adjacent steps in the irregular staircase is more than or equal to +2.
Clause 20. The method of claim 16, wherein the irregular staircase is a non-repeating staircase.
Clause 21. The ophthalmic lens of claim 20, wherein the irregular staircase is an adjacently non-repeating staircase.
Clause 22. The ophthalmic lens of claim 16, wherein the ophthalmic lens comprises a quadrifocal intraocular lens (IOL).
Clause 23. The ophthalmic lens of claim 16, wherein the combined visual acuity score is a sum of visual acuity values for the plurality of focal regions.
Clause 24. The ophthalmic lens of claim 16, wherein the plurality of focal points correspond to a plurality of diffractive orders, wherein the plurality of diffractive orders does not include the first diffractive order.

PARTS LIST

| | |
|---|---|
| 10 | Optical axis |
| 12 | Focal region |
| 20 | Focal region |
| 30 | Focal region |
| 100 | IOL |
| 110 | Haptic |
| 120 | Lens body |
| 130 | Echelette profile |
| 131 | Echelette |
| 132 | Diffractive unit |
| 133 | Phase-shift unit |
| 134 | Staircase step |
| 200 | Flowchart |

It will be understood by those skilled in the art that various changes may be made and equivalents may be substituted without departing from the scope of the novel techniques disclosed in this application. In addition, many modifications may be made to adapt a particular situation or material to the teachings of the novel techniques without departing from its scope. Therefore, it is intended that the novel techniques not be limited to the particular techniques disclosed, but that they will include all techniques falling within the scope of the appended claims.

1. An ophthalmic lens defining an optical axis, the ophthalmic lens comprising:
a plurality of annular regions extending in a radial direction away from the optical axis, including a corresponding plurality of echelettes, wherein the plurality of echelettes forms an echelette profile, wherein the echelette profile includes a staircase profile, wherein each echelette includes a staircase step,
wherein, for each staircase step, there are M phase-shift units arranged vertically, wherein M is an integer, wherein each phase-shift unit has a phase height configured to shift the phase of a reference frequency of light by approximately N*2*π, where N is an integer, and
wherein the staircase profile forms an irregular staircase.

2. The ophthalmic lens of claim 1, wherein a differential in a number of phase-shift units between two adjacent staircase steps in the staircase profile is less than or equal to −2.

3. The ophthalmic lens of claim 1, wherein a differential in a number of phase-shift units between two adjacent staircase steps in the staircase profile is zero.

4. The ophthalmic lens of claim 1, wherein a differential in a number of phase-shift units between two adjacent staircase steps in the staircase profile is more than or equal to +2.

5. The ophthalmic lens of claim 1, wherein the ophthalmic lens comprises an intraocular lens (IOL).

6. The ophthalmic lens of claim 5, wherein the IOL comprises a quadrifocal IOL.

7. The ophthalmic lens of claim 1, wherein the irregular staircase is a non-repeating staircase.

8. The ophthalmic lens of claim 7, wherein the irregular staircase is an adjacently non-repeating staircase.

9. A method for constructing a multifocal intraocular lens (IOL) defining a plurality of focal regions corresponding to focal points for a plurality of diffractive orders, wherein the multifocal IOL includes a plurality of annular regions formed in a medium and extending in a radial direction away from an optical axis, wherein the plurality of annular regions include a plurality of echelettes, wherein each echelette includes a staircase step including M phase-shift units, wherein M is an integer, wherein each phase-shift unit has a thickness approximately equal to N wavelengths for a reference frequency of light, wherein N is an integer, wherein each echelette includes a diffractive unit, wherein the arrangement of the plurality of echelettes defines an echelette profile, wherein the arrangement of the plurality of staircase steps defines a staircase profile, the method comprising:
determining, for each of a plurality of candidate echelette profiles, a performance characteristic for each of the plurality of focal regions, wherein each performance characteristic comprises a visual acuity value;
combining each visual acuity value in each of the given ones of the candidate echelette profiles to form a combined score for each of the candidate profiles;
selecting, from the plurality of candidate echelette profiles, a selected echelette profile according to the combined score for the selected echelette profile; and
fabricating the multifocal IOL using the selected echelette profile.

10. The method of claim 9, wherein at least two of the plurality of candidate echelette profiles have different staircase profiles.

11. The method of claim 10, wherein at least one of the plurality of candidate echelette profiles includes an irregular staircase.

12. The method of claim 11, wherein the selected echelette profile comprises an irregular staircase.

13. The method of claim 12, wherein the irregular staircase is a non-repeating staircase.

14. The ophthalmic lens of claim 13, wherein the irregular staircase is an adjacently non-repeating staircase.

15. The method of claim 9, wherein each combined score is a sum of the visual acuity values for given ones of the candidate echelette profiles.

16. An ophthalmic lens defining an optical axis and a plurality of focal points, the ophthalmic lens comprising:
a plurality of annular regions extending in a radial direction away from the optical axis, including a corresponding plurality of echelettes formed in a medium, wherein the plurality of echelettes form an echelette profile, wherein each echelette includes a staircase step, wherein the plurality of staircase steps define a staircase profile,
wherein each staircase step includes M phase-shift units, wherein M is an integer, wherein each phase-shift unit has a thickness approximately equal N wavelengths for a reference frequency of light through the medium, where N is an integer,
wherein the staircase profile includes an irregular staircase, and
wherein the irregular staircase is selected from a plurality of candidate staircase profiles according to a combined visual acuity score for a plurality of focal regions corresponding to the plurality of focal points.

17. The ophthalmic lens of claim 16, wherein a differential in a number of phase-shift units between adjacent steps in the irregular staircase is less than or equal to −2.

18. The ophthalmic lens of claim 16, wherein a differential in a number of phase-shift units between adjacent steps in the irregular staircase is zero.

19. The ophthalmic lens of claim 16, wherein a differential in a number of phase-shift units between adjacent steps in the irregular staircase is more than or equal to +2.

20. The method of claim 16, wherein the irregular staircase is a non-repeating staircase.

21. The ophthalmic lens of claim 20, wherein the irregular staircase is an adjacently non-repeating staircase.

22. The ophthalmic lens of claim 16, wherein the ophthalmic lens comprises a quadrifocal intraocular lens (IOL).

23. The ophthalmic lens of claim 16, wherein the combined visual acuity score is a sum of visual acuity values for the plurality of focal regions.

24. The ophthalmic lens of claim 16, wherein the plurality of focal points correspond to a plurality of diffractive orders, wherein the plurality of diffractive orders does not include the first diffractive order.
